(19) United States
(12) Patent Application Publication
(10) Pub. No.: US 2007/0213781 A1
(43) Pub. Date: Sep. 13, 2007

(54) RESEALABLE SEAL MEMBER INCLUDING A LUBRICATED PASSAGE AND METHODS RELATED THERETO

(76) Inventors: Benjamin R. Fruland, Plymouth, MN (US); Larry L. Hum, Cottage Grove, MN (US); Lawrence D. Swanson, White Bear Lake, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/276,699

(22) Filed: Mar. 10, 2006

(51) Int. Cl. A61N 1/00

(52) U.S. Cl. 607/37

(57) ABSTRACT

A body implantable retaining assembly for forming an electrical or mechanical connection between a medical device and a lead is discussed herein. A first bore is formed in a housing of the device for receiving a lead terminal portion. A second bore is also formed in the device housing, which opens to the first bore and to an exterior housing surface. A retaining assembly is mounted within the second bore and is positioned to align with a lead terminal pin or a lead terminal ring. The retaining assembly is adjustable by an instrument, thereby allowing engagement and securing of the lead and the housing. A seal member provides electrical and fluid isolation of the retaining assembly from bodily fluids located proximate to the housing exterior surface. The seal member is disposed in the second bore between the retaining means and the exterior housing surface and includes a lubricated passage therethrough.

### TECHNICAL FIELD

This patent document pertains generally to an implantable medical device and its connection with one or more electrical leads. More particularly, but not by way of limitation, this patent document pertains to a resealable seal member including a lubricated passage adapted to admit an instrument therethrough and reliably isolate electrical contacts from bodily fluids in the absence of such instrument.

### BACKGROUND

Pacemakers and other implantable medical devices (referred to as “IMDs”), such as cardiac defibrillators, require a means of passing electrical signals between the device and one or more (relatively remote) portions of a patient's body (e.g., the heart). To transmit these electrical signals from the device to the heart, at least one implantable lead is conventionally used. The at least one lead makes electrical contact with the device on a lead terminal portion through a lead terminal pin or a lead terminal ring(s). After a connection between the lead and the device has been established, the electrical signals may be transmitted from the device to the heart (or other remote portion of the patient's body) via the at least one lead. To ensure an uninterrupted electrical path between the device and the heart (or elsewhere), a strong and reliable connection between the implantable lead and the device is desirable.

Another quality of the interconnection between the device and the at least one lead is that such connection should be detachable after being (previously) attached. Under certain circumstances, the device may need to be removed from the patient while the lead remains in place to be used with a new device. Accordingly, a means for securably connecting the lead with the device should also allow the lead to be removed from the device after a length of time (e.g., one or more years) without damage to the lead.

After establishing a tight and secure connection, bodily fluids should be prevented from invading (i.e., leaking into) the vicinity of the connection and contacting associated electrical contacts. Among other things, the leakage of bodily fluids into or near the connection arrangement may cause corrosion of connector parts or short-circuiting of the electrical signals produced or received by the device. For instance, the short-circuiting of the electrical signals may result in a partial loss of stimulation pulses that are intended to be supplied to the heart or non-physiologic noise, the latter of which may be sensed by the device resulting in inappropriate therapy being delivered. Additionally, due to the short-circuiting, tissue in the region of the interconnection may be undesirably stimulated, which may result in muscle spasms that are uncomfortable for the patient.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic view illustrating a system including an implantable medical device and an electrical lead, and an environment in which the system may be used.

FIG. 2A is a perspective view illustrating a bipolar electrical lead extending from a lead terminal portion to a lead distal portion.

FIG. 2B is a schematic view illustrating a system including an implantable medical device and an electrical lead for delivering or receiving electrical signals to and from a heart.

FIG. 3 is a cross-sectional view of an implantable medical device and a portion of an electrical lead taken along line 3-3 of FIG. 2B.

FIG. 4A is an isometric view illustrating an instrument and a seal member including a non-lubricated passage.

FIG. 4B is an isometric view illustrating a seal member including portions of a non-lubricated passage.

FIG. 4C is a cross-sectional view of a seal member including a non-lubricated passage and an instrument inserted therein taken along line 4C-4C of FIG. 4A.

FIG. 4D is a cross-sectional view of a seal member including a non-lubricated passage and an instrument inserted therein taken along line 4D-4D of FIG. 4A.

FIG. 5A is an isometric view illustrating an instrument and a seal member including a lubricated passage.

FIG. 5B is an isometric view illustrating a seal member including a lubricated passage.

FIG. 5C is a cross-sectional view of a seal member including a lubricated passage and an instrument inserted therein taken along line 5C-5C of FIG. 5A.

FIG. 5D is a cross-sectional view of a seal member including a lubricated passage and an instrument inserted therein taken along line 5D-5D of FIG. 5A.

FIG. 6 is a flow diagram illustrating a method of operatively coupling an electrical lead to an implantable medical device.

### DETAILED DESCRIPTION

The following detailed description includes references to the accompanying drawings, which form a part of the detailed description. The drawings show, by way of illustration, specific embodiments in which the present members, assemblies, systems, and methods may be practiced. These embodiments, which are also referred to herein as “examples,” are described in enough detail to enable those skilled in the art to practice the present members, assemblies, systems, and methods. The embodiments may be combined, other embodiments may be utilized, or structural or logical changes may be made without departing from the scope of the present members, assemblies, systems, and methods. The following detailed description is, therefore, not to be taken in a limiting sense, and the scope of the present members, assemblies, systems, and methods are defined by the appended claims and their legal equivalents.

In this document the terms “a” or “an” are used to include one or more than one; the term “or” is used to refer to a nonexclusive or unless otherwise indicated; and the term “subject” is used to include the term “patient.” In addition, it is to be understood that the phraseology or terminology employed herein, and not otherwise defined, is for the purpose of description only and not of limitation.

Introduction

Due to the often life sustaining functions provided by IMDs, a failure to maintain a connection between a device and a lead (which transmits device-generated therapy to one or more remote portions of a body into which the device is implanted) could be detrimental. Accordingly, it is important that a lead is safely secured to an IMD to prevent it from being inadvertently decoupled therefrom. In addition, instances may exist where it is desirably to detach the lead from the IMD, and thus such secure connection should also be readily detachable. As one example, the IMD may need to be removed and replaced as a complete unit without disturbing the electrode(s) disposed on the lead. After the lead is safely secured to the IMD, it is important that bodily fluids are not allowed to leak into or near the interconnection; as such leakage can cause a partial diversion of energy that may affect the delivery of life sustaining functions provided by the IMD (due to, e.g., electrical shorting) and may further result in irregular sensing by the lead electrodes.

Advantageously, the present members, assemblies, systems, and methods provide a resealable seal member including a lubricated passage, which provides quick and convenient instrument access to a retaining assembly for securing or releasing a terminal portion of a lead and an IMD. In addition, the present members, assemblies, systems, and methods provide liquid tight sealing of the (device and lead) interconnection in an absence of the instrument.

Examples

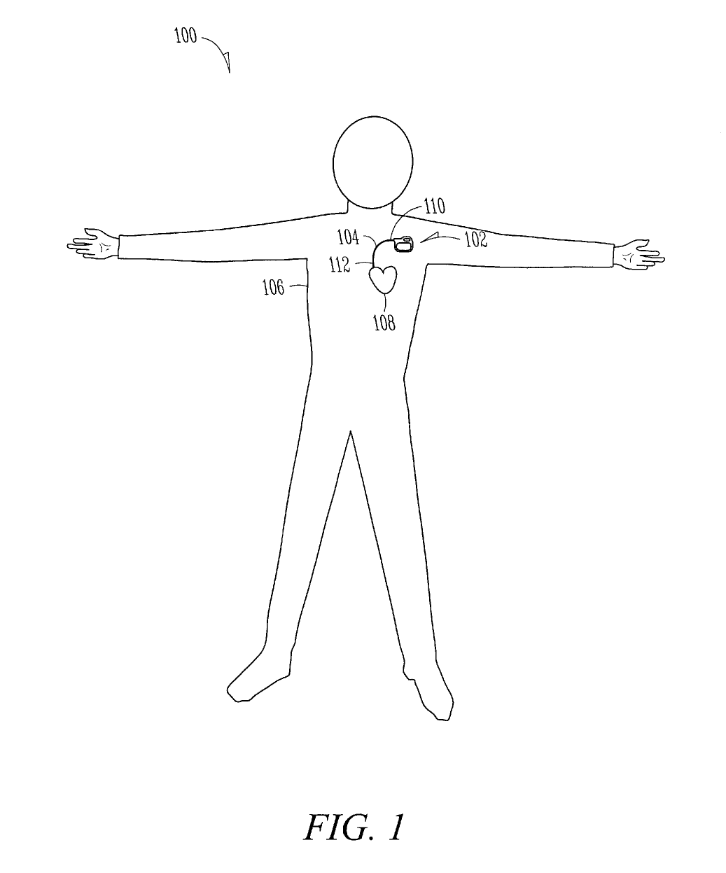

Turning now to the drawings, FIG. 1 illustrates an implantable system 100 and an environment 106 (e.g., a subcutaneous pocket made in a wall of a subject's chest, abdomen, or elsewhere) in which system 100 may be used. In varying examples, system 100 may be used for delivering or receiving electrical signals to stimulate or sense, respectively, a heart 108 of a subject. As shown in FIG. 1, system 100 includes an IMD 102 and an electrical lead 104 extending from a lead terminal portion 110 to a lead distal portion 112. Lead 104 is coupled with IMD 102 at lead terminal portion 110, while lead distal portion 112 is disposed on, about, or within heart 108 thereby electrically connecting the heart and IMD 102.

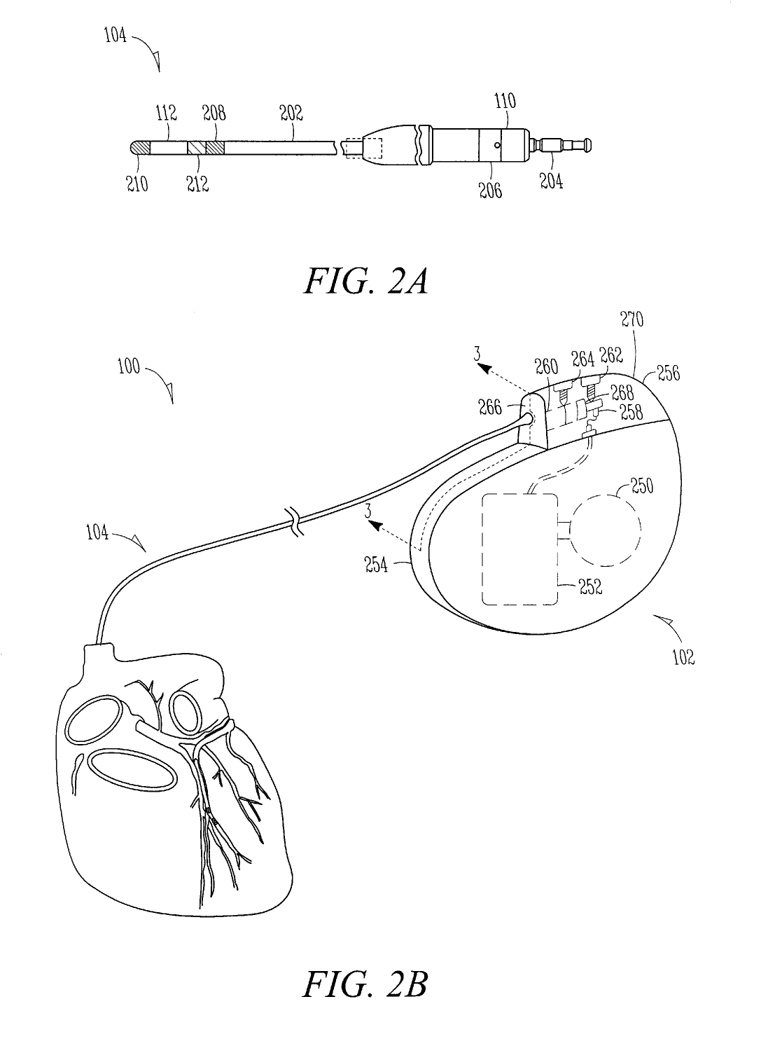

IMD 102 includes a source of power 250 (FIG. 2B) as well as an electronic circuitry portion 252 (FIG. 2B). In this example, IMD 102 is a battery-powered device that senses intrinsic signals of heart 108 and generates a series of one or more timed electrical discharges (e.g., pulses or shocks). IMD 102 generically represents, among other things, cardiac rhythm management devices (referred to as “CRM devices”) such as pacers, cardioverters, biventricular/cardiac resynchronization devices, defibrillators, or sensing instruments.

FIG. 2A illustrates a generic lead 104 including a lead body 202 extending from a lead terminal portion 110 to a lead distal portion 112. In one example, lead body 202 comprises biocompatible tubing such as medical grade polyurethane. In another example, lead body 202 comprises a medical grade silicone rubber or other thermoplastic or polymer suitable for use in leads. As discussed above, system 100 (FIG. 1) includes lead 104 for electrically coupling an IMD 102 (FIG. 1) to remote bodily tissue, such as a heart 108 (FIG. 1) for sensing intrinsic or responsive electrical heart activity or delivering electrical therapy, such as pacing stimulations or defibrillation countershocks, thereto.

In this example, but as may vary, a bipolar lead is shown. The bipolar lead includes two terminal connectors (i.e., a terminal pin 204 and a terminal ring 206) disposed on lead terminal portion 110 and two electrodes 208 and 210 disposed on lead distal portion 112. Terminal pin 204 and terminal ring 206 are electrically coupled to respective electrodes 208 and 210 via at least two conductors contained within lead body 202. In addition or alternative to electrodes 208 or 210 (which are typically adapted to sense or stimulate heart 108), lead distal portion 112 may also includes means for sensing one or more other physiological parameters, such as pressure, oxygen saturation, or temperature, or for the delivery of one of more drugs. As shown, a drug collar 212 adapted to time release one or more drugs to a subject 106 (FIG. 1) may be disposed on lead body 202.

FIG. 2B illustrates a system 100 including an IMD 102 coupled with an implantable lead 104. In this example, IMD 102 includes a housing comprising a can portion 254 and a header portion 256. As shown, can portion 254 includes a source of electrical energy (e.g., a battery) 250 and a device for generating a pulse or sensing heart activity (e.g., electrical circuitry) 252. In one example, can portion 254 comprises a corrosion-resistant metal, such as titanium, which is sealed after battery 250 and electrical circuitry 252 are placed therein.

Header portion 256 is formed on or attached to can portion 254. In one example, header portion 256 comprises an epoxy resin, polyurethane, or similar polymer material, which is formed, molded, or assembled with one or more other members of the header (e.g., an electrical terminal(s) 258). In this example, but as may vary, header portion 256 includes a first bore 260, a second bore 262, and a third bore 264. First bore 260 is typically longitudinally disposed inward from a header front surface 266 and is typically sized and shaped to receive an electrically conductive terminal portion 110 of one or more leads, such as a bipolar lead 104, as illustrated in FIG. 2A. First bore 260 may be referred to as a “stepped bore” because it typically includes one or more steps 268, which transition to reduced diameters proceeding axially inward into header portion 256 from surface 266. As shown, an electrical terminal 258 is typically located at an inboard end of first bore 260 and is electrically connected to electrical circuitry 252.

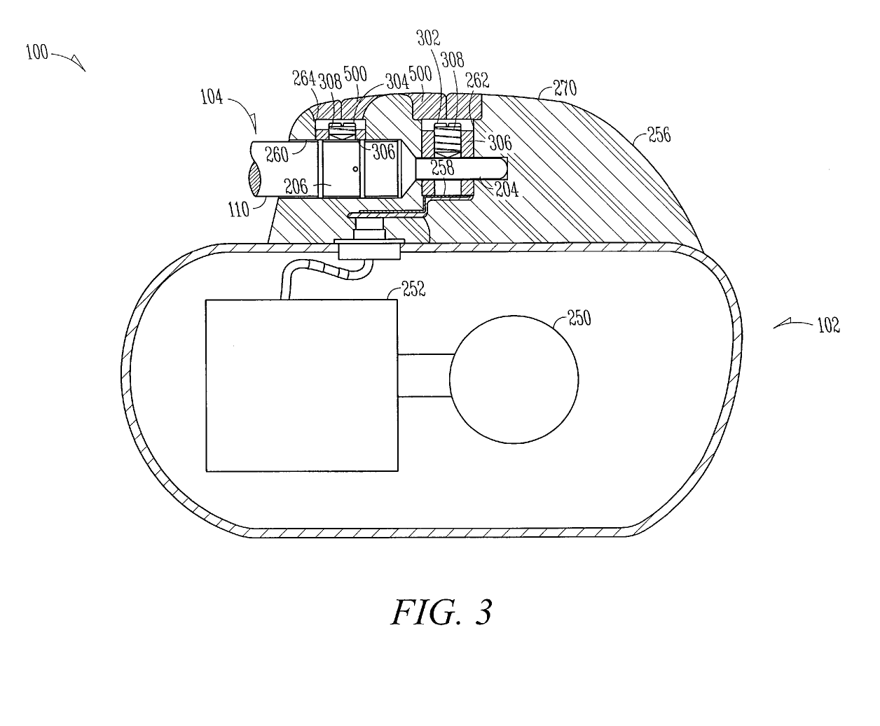

In this example, second 262 and third 264 bores are typically disposed substantially transverse to first bore 260 and open to both the first bore and an exterior surrounding surface 270 of the header 256. Second 262 and third 264 bores are typically positioned to align with respective terminal connectors (e.g., lead terminal pin 204 (FIG. 2A) and lead terminal ring 206 (FIG. 2A)) when lead 104 is (filly) inserted into first bore 260. One or more electrically conductive retaining assemblies 302, 304 (FIG. 3) (e.g., set-screw assemblies) are typically disposed in second 262 and third 264 bores to make a mechanical or electrical connection between lead 104 and IMD 102, as further discussed in association with FIG. 3. Alternatively, while not shown in FIG. 2B, one or more of first 260, second 262, or third 264 bores may be disposed in can portion 254 of the IMD housing rather than header portion 256.

FIG. 3 illustrates a partial cross-sectional view of a system 100 including an IMD 102 mechanically and electrically coupled with an implantable lead 104 via a first 302 and a second 304 retaining assembly. In this example, a terminal portion 110 of lead 104 (including a lead terminal pin 204 and a lead terminal ring 206) is received by a first housing bore 260 in header portion 256. A second 262 and a third 264 housing bore are typically disposed in header portion 256 (traverse to first bore 260), such that the second 262 and third 264 bores align with lead terminal pin 204 and lead terminal ring 206, respectively, when lead terminal portion 110 is inserted into first bore 260.

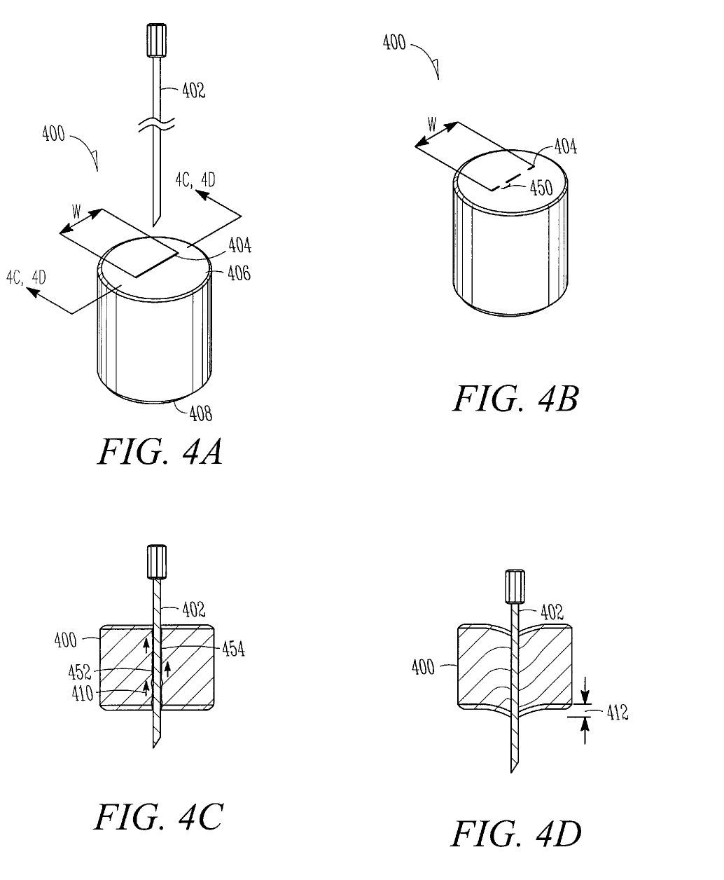

As shown, first retaining assembly 302 is mounted in second bore 262 and second retaining assembly 304 is mounted in third bore 264. The retaining assemblies 302, 304 releasably engage its associated one of lead terminal pin 204 or lead terminal ring 206, such as for a mechanical mounting of lead 104 to header 256. In varying examples, each retaining assembly 302, 304 includes, at least in part, a receptacle 306 and a fastener 308 configured to engage with receptacle 306. In one example, but as may vary, each receptacle 306 includes one or more internal threads and each fastener 308 includes one or more external threads conforming to the internal threads. In such an example, an instrument 402 (FIG. 4A) (such as a screwdriver or Allen wrench) may be brought into engagement with each fastener 308 and rotated to effectuate a coupling or decoupling of lead 104 and IMD 102. For instance, each fastener 308 may be turned in a first direction to advance its tip downwardly into the respective bore establishing a connection between lead 104 and IMD 102 or may be turned in a second direction to retract its tip from such bore effectuating a decoupling of lead 104 and IMD 102.

In the example shown in FIG. 3, an advancement of fastener 308 into second bore 262 results in an electrical coupling of lead terminal pin 204 with an electrical terminal 258 of header 256. In this way, electrical signals may be communicated between lead 104 and electrical circuitry 252 of the IMD. Although not shown, a further electrical terminal may be disposed adjacent lead terminal ring 206 allowing electrical coupling therewith, thereby allowing further communication between lead 104 and electrical circuitry 252.

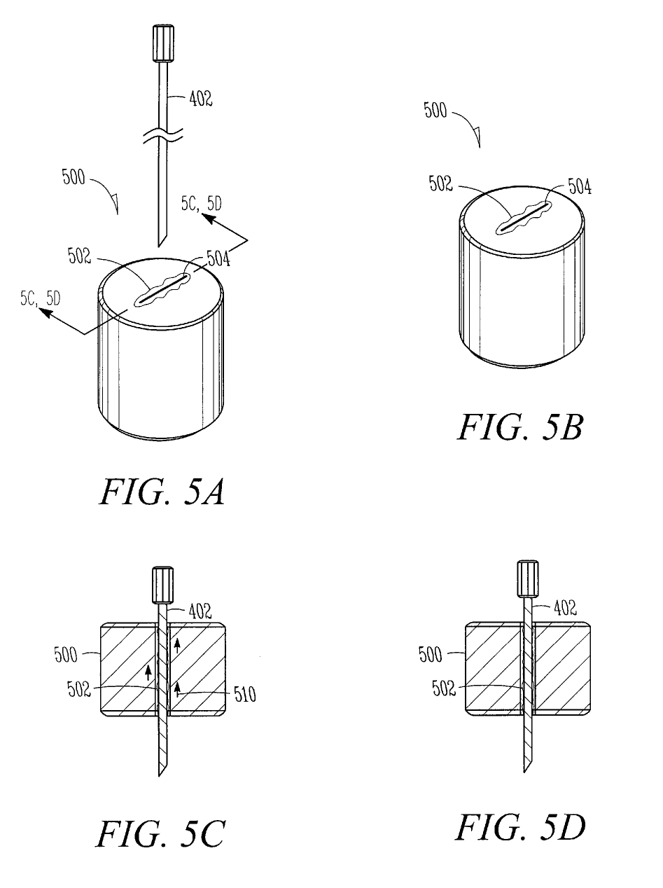

Due to the fact that IMD 102 is exposed to bodily fluids, a reliable seal for sealing first 262 and second 264 bores is desired to electrically isolate retaining assemblies 302, 304 and protect the interconnection between lead 104 and IMD 102 from exposure to the bodily fluids. To allow for the actuation (e.g., the rotation) of a portion of the retaining assemblies 302, 304, the seal should allow an instrument to be inserted therethrough as desired by a caregiver. Advantageously, present seal member 500 (see also, FIG. 5A) allows for the passage of an instrument therethrough, while further being configured to substantially seal second 262 and third 264 bores (liquid tight) in the absence of the instrument.

As shown, seal member 500 is typically disposed in second 262 and third 264, such as bores between each bore's respective retaining assembly (i.e., 302, 304) and an exterior surface 270 of header 256. In one example, but as may vary, sealing member 500 includes a circular shape having a diameter at least as large as a diameter of the second 262 or third 264 bores. In such an example, seal member 500 may be compressively secured in one of the bores. In another example, a biocompatible medical adhesive is used to secure seal member 500 in a respective one of the bores 262 or 264.

Seal member 500 helps provide electrical and fluid isolation of retaining assemblies 302, 304 from bodily fluids located proximate to exterior surface 270 of the IMD housing. A lubricated passage 504 therethrough promotes low shear insertion of instrument 402 (FIG. 4A) into bores 262, 264 for operable engagement with portions of a retaining means (e.g., fastener 308). Upon withdrawal of instrument 402, seal member 500 returns to a substantially liquid tight sealing configuration (without rebonding between seal member halves) to close passage 504, thereby providing electrical and fluid isolation to the retaining assemblies.

As illustrated in FIGS. 5A-5D and discussed in the text associated therewith, the addition of one or more lubricants to seal member 500, specifically passage 504 of the seal member, provides many advantages over non-lubricated seal members 400 (see, FIGS. 4A-4D). As one example, the addition of a lubricant 502 (FIG. 5A) (e.g., a biocompatible or fluorosilicone lubricant) to seal member 500 typically increases the reliability of such seal member by, among other things, decreasing a coefficient of friction between seal member 500 and any instrument 402 (FIG. 4A) that might be inserted therethrough. The reduction of friction reduces the shear stress felt by seal member 500 and accordingly, decreases a deflection amount 412 (FIG. 4D) of seal member 500 upon instrument 402 insertion. As another example, the addition of one or more lubricants to seal member 500 puts a dissimilar material between the two (e.g., silicone) seal member halves and prevents them from rebonding or sticking together, which may otherwise result in seal member 500 damage upon instrument 402 (re)insertion.

FIGS. 4A-4D illustrate one or both of a non-lubricated seal member 400 (i.e., a seal member with a non-lubricated passage 404) and an instrument 402 used to, among other things, engage with portions of a retaining assembly 302 or 304 (FIG. 3) for actuation thereof. As shown in FIG. 4A, non-lubricated seal member 400 includes a passage 404 providing access to retaining assembly 302 or 304 by instrument 402. In varying examples, passage 404 extends from a seal member first end surface 406 to a seal member second end surface 408 and comprises an (initial) width W. When non-lubricated seal member 400 is contained within a bore (e.g., first bore 262 or second bore 264) of an IMD 102 and not invaded by instrument 402, passage 404 is closed as shown providing both fluid and electrical isolation of retaining assembly 302 or 304 (FIG. 3) from the environment about the IMD housing.

Non-lubricated seal member 400, like lubricated seal member 500 (e.g., a seal member including a lubricated passage 504 shown in FIG. 5A), is deformable in the region about passage 404 to admit instrument 402 through the seal member and into an operative engagement with, for example, a fastener 308 (FIG. 3) of a retaining assembly 302 or 304. However, unlike lubricated seal member 500, non-lubricated seal member 400 has at least three drawbacks associated with its use. FIGS. 4B-4D each illustrate a mode in which insertion of instrument 402 into passage 404 of non-lubricated seal member 400 may compromise the functionality (e.g., the fluid and electrical isolation ability) of the seal and thus the IMD 102 in which it is provided.

In FIG. 4B, a non-lubricated seal member 400 including a passage 404 is illustrated. In one example, the non-lubricated seal member 400 comprises silicone or other similar materials. In such an example, the creation of passage 404 having width W results in a silicone passage first surface and a silicone passage second surface in compressive or other intimate contact, which over time can rebond (e.g., via hydrogen or covalent bonding) together. As a result, portions of passage width W become nonexistent, such as is shown. When instrument 402 then attempts to pass through passage 404, the non-lubricated seal member 400 can become damaged due to, for example, tearing. In one example, the tearing may occur along width W of the passage, such as at or near the rebonded passage width W portions 450. In another example, the tearing may occur at one or both edges of the passage.

FIG. 4C-4D are cross-sectional views of a non-lubricated seal member 400 and an instrument 402 taken along line 4C-4C and 4D-4D of FIG. 4A. As shown in FIG. 4C, when passing instrument 402 through a non-lubricated seal member 400, the frictional shear forces 410 may be high enough to damage one or both of the passage first surface 452 or the passage second surface 454 (e.g., due to frictional wearing of such surfaces). In addition to wearing of the passage surface(s) 452 or 454, the friction of instrument 402 passing through non-lubricated seal member 400 may cause the seal member to deflect by an amount 412, thereby pinching or “bottoming-out” on any underlying material or member and becoming damaged.

In brief, without the use of one or more lubricants applied to a passage of a seal member, portions of the seal member may rebond to one another. Additionally, the lack of a lubricant may result in a high shear stress present between an inserted instrument and a seal member causing, among other things, wearing of a surface of the seal member or deflection of one or more seal member portions, all of which may jeopardize the sealing ability of the seal member upon removal of the instrument.

The addition of one or more biocompatible or fluorosilicone lubricants 502 to a passage 504 of a seal member 500 increases the reliability of the seal system when breached by an instrument 402 (due to little or no seal member damage). According to one example, but as may vary, the lubricant is applied by inserting a tool (e.g., a rod) coated with lubricant through passage 504 either manually or with an automated system. FIGS. 5A-5D illustrate one or both of a lubricated seal member 500 (i.e., a seal member with a lubricated passage 504) and an instrument 402. Examples of lubricants 502 that may be used with seal member 500 include MDX4-4159, NuSil MED420, MED-4159, MED-4162, MED1-4162, all of which are manufactured by Dow Coming® headquartered in Midland, Mich., USA or NuSil Technology® headquartered in Carpinteria, Calif., USA. Other lubricants or liquids that may reduce friction between seal member 500 and instrument 402 or prevent portions of the seal member from rebonding are also within the scope of the present members, assemblies, systems, and devices.

As shown in FIG. 5B, the addition of a biocompatible or fluorosilicone lubricant 502 to passage 504 places a (thin) layer of dissimilar material between the intimate contacting seal member 500 halves precluding any rebonding between the same. As shown in FIGS. 5C-SD, the addition of lubricant 502 to portions of passage 504 reduces the friction 510 between instrument 402 and seal member 500 and further reduces or eliminates deflection of the seal member (see, contra, FIG. 4D).

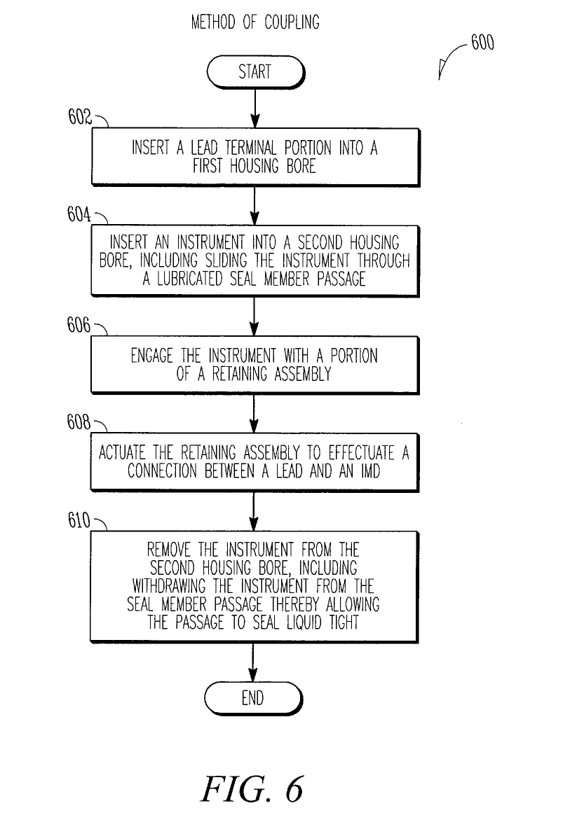

FIG. 6 is a flow diagram illustrating a method 600 of operatively coupling at least one lead to a housing of an implantable medical device. At 602, a lead terminal portion is inserted into a first housing bore such that at least one terminal connector (e.g., a lead terminal pin or a lead terminal ring) is aligned with a second housing bore and a retaining assembly contained therein. In one example, the lead terminal portion is inserted into the first housing bore until a lead terminal pin is aligned with a proximal retaining assembly (contained in the second bore) or a lead terminal ring is aligned with a distal retaining assembly (contained in a third bore). At 604, an instrument (e.g., a tip and shaft of a screwdriver) is inserted into the second bore. The insertion of the instrument into a housing bore includes inserting the instrument through a lubricated passage of a seal member. In one such example, the passage is lubricated using one or both of a biocompatible lubricant or a fluorosilicone lubricant.

At 606, the instrument is engaged with a portion of the retaining assembly (e.g., a fastener) contained in the second bore and manipulated (e.g., rotated) to actuate the retaining assembly at 608. In one example, manipulation of the instrument in a first direction effectuates a coupling between the lead and the device housing. A coupling of the lead and device housing secures the lead mechanically within the housing, as well as establishes an electrical connection between electrical circuitry of the IMD and the lead. Should the need arise to adjust the positioning of the lead within the housing or to remove the lead or IMD for inspection or replacement, release of the lead from the IMD may be accomplished by manipulation of the instrument (at 608) in a second direction. At 610, the instrument is removed from the second housing allowing the lubricated passage of the seal member to seal in a liquid tight manner. The instrument may be similarly inserted into another housing bore to actuate the associated retaining assembly disposed therein.

Conclusion

Among other things, a body implantable retaining assembly for forming an electrical and mechanical connection between an implantable medical device and one or more leads is discussed. The retaining assembly is mounted within a housing of the device (e.g., a header portion) and is positioned to align with a lead terminal pin or a lead terminal ring when the lead is inserted into a portion of the device. The retaining assembly is adjustable by an instrument to engage or release the lead and the device. A seal member provides electrical and fluid isolation of the retaining means from bodily fluid located proximate to an exterior surface of the device housing. The seal member includes a lubricated passage therethrough, which is elastically deformable to allow (low shear) insertion of the instrument into an operable engagement with the retaining assembly. Upon withdrawal of the instrument, the seal member returns to a sealing configuration (without rebonding between one or more portions of the seal member) to close the passage and provide electrical and fluid isolation to the interconnection of the device and one or more leads.

It will be appreciated by those skilled in the art that the teachings associated with the present seal member (i.e., including a lubricated passage) may be applied to all seals including one or more passages that are breached by, for example, an instrument and which must be sealed upon the removal of the instrument. For instance, the seal member may be used in systems for pacing, cardioversion/ defibrillation, neuromuscular stimulation, bone growth stimulation and the like. Additionally, although the present seal member has been described with reference to particular materials, sizes, and shapes; any suitable size, shape, or materials may be used without departing from the scope of the invention discussed herein.

While FIG. 2A illustrates a bipolar lead and FIGS. 2B and 3 illustrate and describe a system including a bipolar lead, the present subject matter is not so limited. Unipolar leads (i.e., leads including one terminal connector and one corresponding distal electrode) and other multipolar leads (i.e., leads including more than two terminal connectors and corresponding distal electrodes) are also within the scope of the present members, assemblies, systems, and devices. Similarly, one or multiple (more than two) bores, retaining assemblies, or seal members including a lubricated passage may be also used in the system.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the present assemblies and methods should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

What is claimed is:

1. A member for providing electrical and fluid isolation in an implantable medical device, the member comprising:
a resilient seal including at least one passage extending therethrough from a seal first end surface to a seal second end surface, each passage forming a passage first surface and a passage second surface;
one or more lubricants disposed on one or both of the passage first surface or the passage second surface;
wherein the at least one passage and the one or more lubricants are configured to slidably receive an instrument and close a bore liquid tight in an implantable medical device housing in the absence of the instrument; and
wherein the resilient seal is sized and shaped to be retained in the bore.

2. The member as recited in claim 1, wherein an outer diameter of the resilient seal is at least as large as a diameter of the bore.

3. The member as recited in claim 1, wherein the resilient seal comprises silicone rubber.

4. The member as recited in claim 1, wherein the one or more lubricants comprise a biocompatible lubricant.

5. The method as recited in claim 1, wherein the one or more lubricants comprise a fluorosilicone lubricant.

6. A system for establishing an electrical and mechanical connection between an implantable medical device and a lead, the system comprising:
a device housing including at least a first bore and a second bore, the first bore sized and shaped to receive a terminal portion of the lead and the second bore open to the first bore and to an exterior surface of the device housing;
a retaining assembly mounted, at least in part, within the second bore and positioned to align with a terminal connector when the lead terminal portion is inserted into the first bore, the retaining assembly being adjustable by an instrument;
a seal member disposed in the second bore between the retaining assembly and the housing exterior surface, the seal member including a lubricated passage therethrough and being deformable to slidably allow the instrument through the passage and into an operable engagement with the retaining assembly; and
wherein the seal member is configured to seal the passage liquid tight in the absence of the instrument.

7. The system as recited in claim 6, wherein the device housing includes a can portion and a header portion, the first and second bores formed within the header portion.

8. The system as recited in claim 6, wherein the retaining assembly comprises, at least in part, a fastener and a receptacle configured to engage with the fastener.

9. The system as recited in claim 8, wherein the receptacle comprises one or more internal threads and the fastener comprises one or more external threads conforming to the internal threads; and
wherein the instrument is configured to rotate the fastener.

10. The assembly as recited in claim 6, wherein the terminal connector comprises a lead terminal pin or a lead terminal ring.

11. The system as recited in claim 6, wherein the seal member comprises a circular shape, the circular shape having a diameter at least as large as a diameter of the second bore.

12. The system as recited in claim 6, wherein an axial orientation of the second bore is substantially transverse to an axial orientation of the first bore.

13. The system as recited in claim 6, further comprising:
at least a third bore substantially parallel with, and spaced apart from, the second bore;
a further retaining assembly mounted within the third bore and positioned to align with a further terminal connector when the lead terminal portion is inserted into the first bore, the further retaining assembly being adjustable by the instrument;
a further seal member disposed in the third bore between the further retaining assembly and the housing exterior surface, the further seal member including a lubricated passage therethrough and being elastically deformable to slidably allow the instrument through the passage and into an operable engagement with the further retaining assembly; and
wherein the further seal member is configured to seal the passage liquid tight in the absence of the instrument.

14. The system as recited in claim 6, wherein the lubricated passage of the seal member includes a biocompatible lubricant.

15. The system as recited in claim 6, wherein the lubricated passage of the seal member includes a fluorosilicone lubricant.

16. The system as recited in claim 6, wherein the seal member comprises silicone rubber.

17. A system comprising:
a lead extending from a lead terminal portion to a lead distal portion, the lead terminal portion including at least one terminal connector electrically coupled to one or more electrodes disposed on the lead distal portion;
an implantable medical device including a device housing, the device housing comprising:
at least one circuit adapted to deliver or receive one or more electrical signals to stimulate or sense;
a source of electrical energy coupled with the at least one circuit;
a first bore sized and shaped to receive the lead terminal portion;
at least a second bore open to the first bore and to an exterior surface of the housing;
an electrically conductive retaining assembly mounted within the second bore, the electrically conductive retaining assembly including a receptacle positioned to align with the at least one terminal connector when the lead terminal portion is inserted into the first bore and a fastener engagable with the receptacle;
a seal member disposed in the second bore between the electrically conductive retaining assembly and an exterior surface of the housing, the seal member including a lubricated passage therethrough and being deformable to allow insertion of an instrument into the lubricated passage and into an operable engagement with the fastener; and
wherein the fastener is adjustable by movement of the instrument.

18. The system as recited in claim 17, wherein the seal member is adapted to form a liquid tight sealing configuration in the absence of the instrument.

19. The system as recited in claim 17, wherein the lubricated passage comprises a biocompatible lubricant.

20. The system as recited in claim 17, wherein the lubricated passage comprises a fluorosilicone lubricant.

21. A method of operatively coupling at least one lead to a housing of an implantable medical device, the method comprising:
inserting a lead terminal portion into a first housing bore, including aligning at least one terminal connector with a second housing bore;
inserting an instrument into the second housing bore, including sliding the instrument through a seal member passage comprising one or more lubricants;
engaging the instrument with a retaining assembly mounted in the second housing bore;
actuating the retaining assembly to effectuate a connection between the at least one lead and the housing; and
removing the instrument from the second housing bore, including withdrawing the instrument from the seal member passage thereby allowing the passage to seal liquid tight.

22. The method as recited in claim 21, wherein sliding the instrument through the seal member passage includes sliding the instrument through a biocompatible lubricant.

23. The method as recited in claim 21, wherein sliding the instrument though he seal member passage includes sliding the instrument through a fluorosilicone lubricant.

24. The method as recited in claim 21, wherein engaging the instrument with the retaining assembly includes engaging the instrument with a fastener.

25. The method as recited in claim 24, wherein actuating the retaining assembly includes manually imparting a rotational movement to the fastener using the instrument.

26. The method as recited in claim 21, further comprising:
inserting the instrument into a third bore substantially parallel with, and spaced from, the second bore, including sliding the instrument through a further seal member passage comprising one or more lubricants;
engaging the instrument with a further retaining assembly mounted in the third housing bore;
actuating the further retaining assembly to effectuate a further connection between the at least one lead and the housing of the implantable medical device; and
removing the instrument from the third housing bore, including withdrawing the instrument from the further seal member passage thereby allowing the further passage to seal liquid tight.

27. A method of manufacturing a seal member for providing electrical and fluid isolation of a retaining assembly from bodily fluid proximate an exterior surface of an implantable medical device housing, the method comprising:
forming at least one passage through the seal member, including forming a passage extending from a seal first end surface to a seal second end surface;
applying a biocompatible or fluorosilicone lubricant to the seal member passage; and
wherein forming the at least one passage and applying the lubricant provide a means to allow an instrument to engage with the retaining assembly and seal a housing bore liquid tight in the absence of the instrument.

28. The method as recited in claim 27, wherein forming the passage includes forming a passage first surface and a passage second surface.

29. The method as recited in claim 28, wherein applying the lubricant includes applying the lubricant to one or both of the passage first surface or the passage second surface.

30. The method as recited in claim 27, wherein applying the lubricant includes inserting a tool coated with the lubricant through the seal member passage manually or using an automated system.
